SEQUENCE LISTING (3 sequences)

SEQ ID NO: 1 - DNA, 18 bp, Artificial Sequence
  Artificially synthesized oligonucleotide primer
catgggaattcccatgag

SEQ ID NO: 2 - DNA, 33 bp, Artificial Sequence
  Artificially synthesized oligonucleotide primer
ggcgctgcgaattccatggcgcatggcgtccgg

SEQ ID NO: 3 - DNA, 33 bp, Artificial Sequence
  Artificially synthesized oligonucleotide primer
gggggggaattctcactctgatctatccctgtc

(19) United States
(12) Patent Application Publication
(10) Pub. No.: US 2011/0293656 A1
(43) Pub. Date: Dec. 1, 2011

(54) RECOMBINANT MVA CAPABLE OF EXPRESSING STRUCTURAL HCV ANTIGENS

(76) Inventors: Gerd Sutter, Munchen (DE); Volker Erfle, Munchen (DE); Caroline Staib, Munchen (DE); Yuan Wang, Shanghai (CN); Guangdi Li, Shanghai (CN); Li-Xin Zhu, Berkeley, CA (US)

(21) Appl. No.: 13/110,471

(22) Filed: May 18, 2011

Related U.S. Application Data
(63) Continuation of application No. 12/476,696, filed on Jun. 2, 2009, now abandoned.
Application No. 12/476,696 is a continuation of application No. 10/488,301, filed on Aug. 30, 2004, now abandoned.

(30) Foreign Application Priority Data
Sep. 5, 2001 (DE) 101 43 490.1

(51) Int. Cl. A61K 39/285; C07H 21/04; C12N 5/10; A61P 31/18; A61P 31/12; C12N 7/01; A61K 35/76; C07H 21/02

(52) U.S. Cl. 424/199.1; 424/93.2; 536/23.72; 435/235.1; 435/349; 435/352; 435/325

(57) ABSTRACT

The invention relates to recombinant MVA which is capable of expressing structural HCV antigens, functional parts of said structural antigens or epitopes of said structural antigens. The invention further relates to a pharmaceutical composition, especially in the form of a vaccine and containing the recombinant MVA according to the invention, to eukaryotic cells that contain the inventive recombinant MVA and to various uses of the recombinant MVA, for example for producing recombinant structural proteins, for producing a pharmaceutical preparation that is suitable for the therapy and prophylaxis of HCV infections and diseases thereby caused. The invention further relates to methods for producing recombinant MVA and recombinant structural HCV polypeptides encoded by said recombinant MVA, and to DNA or RNA of said recombinant MVA.

### CROSS REFERENCE TO RELATED APPLICATIONS

This application is a continuation of U.S. patent application Ser. No. 12/476,696, filed Jun. 2, 2009; which is a continuation of U.S. patent application Ser. No. 10/488,301, filed Mar. 1, 2004 (now abandoned); which was a National Stage application of PCT/EP02/09958, filed Sep. 5, 2002; which claimed priority to German Patent Application DE 10143490.1, filed Sep. 5, 2001. The disclosure of each of these applications is incorporated by reference herein in its entirety.

### TECHNICAL FIELD

The invention relates to recombinant MVA capable of expressing structural HCV antigens, functional parts of said structural antigens or epitopes of said structural antigens. Furthermore, the invention describes a pharmaceutical composition which is in particular present in form of a vaccine, and a recombinant MVA according to the present invention, eukaryotic cells containing the recombinant MVA of the present invention as well as different applications of the recombinant MVA, e.g. for the production of recombinant structural proteins, for the preparation of a pharmaceutical composition, which is particularly suited for the therapy and prophylaxis of HIV infections and diseases caused thereby, methods for the preparation of recombinant MVA and of recombinant structural HCV polypeptides encoded by said recombinant MVA, and DNA or RNA of said recombinant MVA.

### BACKGROUND

The Hepatitis C virus (HCV) is a positive strand RNA virus belonging to the family of the Flaviviridae. It is the main agent of a non-A-, non-B-hepatitis acquired after a transfusion and in the population (8, 35). More than 70% of the infected patients develop a chronic hepatitis with the risk, that this further develops into a cirrhosis and to a hepatocellular carcinoma (30, 53). The current treatment methods are limited (39, 40, 31, 24, 13). A lot of efforts have been spent to develop vaccines, and several encouraging results have been achieved (9, 41, 22, 25), however no efficient and/or therapeutic vaccine is available up to now.

The structural HCV proteins, e.g. the viral capsid protein nucleus, and the envelope glycoproteins E1 and E2 are promising vaccine targets: The Core antigen is highly conserved in different HCV genotypes and subtypes (6). Internal antigens such as nucleocapsids are accompanied by protecting immune responses (14, 33, 62) in different models, e.g. Rabies Virus, Hepatitis B-Virus and Influenza-Virus. With the following different approaches HCV nucleospecific antibody and CTL responses could be induced and detected (23, 32). In contrast, the core protein seems to have many regulatory functions involved in the modulation of host cell apoptosis, transcription, transformation and immune presentation (43, 4). The immunization of chimpanzees with recombinant E1 und E2 proteins induces a protection against a homologous challenge, presumably via the induction of neutralizing antibodies (9). According to a number of reports the hypervariable domain (HVR) present at the N-terminus of E2 could be an important neutralizing site (20, 34, 67, 68).

The absence of a suitable animal model substantially complicates the study of host immune reactions against HCV infection and the evaluation of a vaccination protection. The absence of an efficient in vitro cell culture system for a productive HCV amplification and minor amounts of HCV particles in infected liver tissues or blood obstructed the formation and the evaluation of more conventional vaccines, e.g. on the basis of live attenuated or inactivated viruses. The use of vector vaccines on the basis of live carrier viruses for the expression of antigen encoding sequences provides an alternative approach in the HCV vaccine development.

### SUMMARY

It is an object of the present invention to provide a novel agent which is suited for prophylaxis and therapy of humans and animals against HCV infections and which avoids the above described disadvantages.

According to the present invention, this object is solved by the recombinant MVA which has thus been modified, that it is able of expressing structural HCV antigens, functional parts of structural HCV antigens and or epitopes of said HCV structural antigens, preferably surface epitopes.

Preferred embodiments of the invention may be taken from the following description as well as from the included claims.

According to the present invention, it has been shown, that recombinant MVA with DNA-Sequences encoding structural HCV antigens, functional parts thereof or epitopes of said structural HCV antigens is excellently suited for the preparation of vaccines. This was surprising insofar as in particular the production or the co-production of the HCV core antigen does not affect the immunogenicity of the vaccine. To be precise, the publication (36) showed, that the immune response in an infected or vaccined host has been suppressed in a specific manner by a replication competent vaccinia virus. Thus, one can assume that such an immune suppression occurs also with the use of MVA as a vector for the production of structural HCV antigens. Unexpectedly it has been shown, however, that with the use of MVA vectors said immune suppression is again compensated. This astonishing experimentally supported result provides an excellent basis for the development of vaccines, in particular on the basis of the HCV antigen as a vaccination antigen.

The viral vector employed according to the present invention is based on the modified vaccinia virus Ankara (MVA), a highly attenuated vaccinia virus strain which has proven to be safe for the clinical use most probably due to its replication deficiency (42, 45, 60, 50). Recombinant MVA (rMVA) as a Vaccine has proven to be efficient in excitating T-cell and antibody responses to different heterologous antigens and to be protecting in the test in animal challenge models for infection diseases in humans, including influenza, AIDS, measles and malaria (61, 3, 27, 52, 15, 59, 1).

Despite of the use known per se of recombinant MVA for the preparation of a vaccine the use of a vaccine in combination with the structural HCV antigens, e.g. the core antigen of HCV as well as E1 and E2 was not obvious in the present case.

The structural antigens of HCV are known per se. These include the capsid protein Core, the envelope glycoprotein E1, and the envelope glycoprotein E2.

It is understood, that it is not necessary to completely express the structural antigens of HCV for the preparation of a vaccine. The experimenter is also able to express only portions of the structural antigens according to the particular experimental schedule and the desired result, in particular naturally functional portions or epitopes of the HCV structural antigens. For example, functional portions include immunogenic portions of the structural HCV antigens. Examples for this include the C-terminal core protein region (aa 127-190 of the HCV polyprotein), the E1 protein region of the HCV Polyprotein as 220-371), as well as the 27 as comprising hypervariable region 1 at the N-terminus of the glycoprotein E2 (Rehermann & Chisari 2000, Curr Top. Microbiol. Immunol 242: 299-325, Penin et al. 2001 J Virol 75: 5703-5710). The functional portions of the structural antigens include e.g. the 124 N-terminal as of the HCV core protein (Kunkel et al., J. Virol 75: 2119-2129, 2001) which are sufficient for the assembly of the virus-like nucleocapsid particles, and the transmembrane domains TMD-E1 as 350-370 or TMD-E2 as 717-740 which are essential for the heterodimerization of the envelope proteins E1 and E2 (Op de Beeck et al., 2000, J. Biol. Chem. 275: 31428-31437) or secretable forms of glycoproteins, which are able to pass the endoplasmic reticulum. Preferably, this is achieved by altering the transmembrane region of E1 or E2 via deletions or other mutations in such a manner, that the proteins are no longer retained in the membrane (Michalak et al., 1997, J Gen Virol 78: 2299-2306).

Epitopes of structural HCV antigens are known. These include e.g. the MHC-I restricted T-cell epitopes core as 2-10, core as 35-44, core as 41-49, core as 85-98, core as 132-140, core as 167-176, core as 178-187, core as 181-190, E1 as 220-227, E1 as 233-242, E1 as 363-371, E2 as 401-411, E2 aa, 489-496, E2 as 521-628 or E2 as 725-733 (Rehermann & Chisari 2000, Curr Top Microbiol Immunol 242: 299-325). Further examples include the E1 epitope as 312-326. The epitopes of the structural HCV antigens may also be determined by techniques known per se, which are for example described in Koziel et al., 1995, J. Clin Invest 96: 2311-2321.

In a preferred embodiment of the present invention the recombinant MVA contains DNA sequences encoding E1 and E2. In particular, it is advantageous to use the E1 antigen in combination with a truncated form of the E2 antigen, so that both antigens are co-produced. “Truncated” means that the lipophilic portion of the E2 glycoprotein is deleted. It has been shown that in this embodiment of the recombinant MVA after vaccination the cellular immune responses directed against the E1 antigen, i.e. the induction of E1-specific cytotoxic T-cells, was markedly increase in comparison to the vaccination with recombinant MVA, which expressed the E1 antigen together with the complete E2 protein. It is understood that it is also possible to arrange different HCV structural antigens, in particular E1 and E2, not on one MVA vector but at least on two vectors. As a result, this also leads to a co-production of structural HCV antigens.

According to the present invention mutation is meant to be a deletion base substitution or a base addition. Chemical modifications are also possible.

The arrangement of structural HCV antigens in recombinant MVA has to be done in such a manner that a functional connection of the coding sequences with regulating elements, e.g. promoters and enhancers is formed. Suitable DNA cloning techniques are available for those skilled in the art. Herein reference may be made e.g. to Sambrook, J. et al., 1989, Molecular Cloning: A Laboratory Manual, Cold Spring Harbor Laboratory, NY; or Ausubel, F. M. et al., 1989, Current Protocols in Molecular Biology, John Wiley & Sons, New York, N.Y. Herein, to said publications exemplarily mentioned reference is made. In particular, the coding DNA sequences have to be arranged and selected in such a manner, that a correct processing of the polypeptides is done in the cells infected with the MVA-HCV vector virus.

The integration of foreign DNA sequences into the MVA genome is preferably done in those regions which are not essential for the replication and infection ability of the MVA. For example, as integration sites natural deletions in the MVA genome are possible. These deletions which do not essentially influence the life cycle of the virus include the deletions I, II, III, IV, V and VI, preferably deletions II and III (Meyer, H., Sutter, G., and Mayr, A., 1991, J. Gen. Virol. 72, 1031-1038). The recombinant MVA thus prepared is infectious, i.e. it is able to infect foreign cells and to express the integrated foreign DNA sequence of HCV. Such recombinant viruses are suitable as extremely safe live vaccines for the treatment or prophylaxis of diseases caused by HCV.

The recombinant MVA according to the present invention may be prepared as generally illustrated in the following. A detailed description may be found in the examples. The insertion of HCV structural antigens into the MVA DNA is done e.g. in the course of a homologous recombination, wherein at both ends the sequence to be introduced is supplied with such sequences which may be recombined with sequences which are arranged nearby naturally occurring deletions, e.g. deletion II or III, in the MVA genome (Antoine et al. 1998, Virology 244: 365-396).

Such a DNA construct which contains a structural HCV antigen or functional parts or epitopes hereof, flanked by MVA-DNA sequences occurring near sites of deletions, e.g. deletion II or III in the MVA genome, is introduced into cells infected with MVA, to enable a homologous recombination. The inserted DNA construct may be linear or circular. Preferably, a circular DNA molecule, in particular a plasmid is inserted.

For the expression of the DNA sequence encoding the HCV structural antigens, regulatory sequences are necessary, which enable a transcription of the DNA sequence. Such regulatory sequences, e.g. promoters and enhancers, are known per se. These include for example also promoters of the vaccinia 11 kDa gene (c.f. EP 0 198 328), of the 7.5 kDa gene (c.f. EP 0 110 385) or synthetic vaccinia virus specific promoters (c.f. Sutter et al., Vaccine 1994, 12:1032).

The DNA construct may be inserted into MVA-infected cells by methods known per se, for example in the course of a transfection by calcium phosphate precipitation (Graham et al., Virol. 52, 456-467, 1973; Wigler et al., Cell 777-785, 1979), by electroporation (Neumann et al., EMBO J. 1, 841-845, 1982), by microinjection (Graessmann, et al., Meth. Enzymology 101, 482-492, 1983), by liposome technology (Straubinger et al., Methods in Enzymology 101, 512-527, 1983), by sphaeroblast technology (Schaffner, Proc. Natl. Acad. Sci. USA 77, 2163-2167, 1980) or by other methods known per se. Preferably, the calcium phosphate technique is employed.

After insertion of the DNA construct into the eukaryotic cells and subsequently employed recombination of the HCV DNA with viral DNA, the recombinant MVA may be isolated in a manner known per se, preferably by means of a marker gene (c.f. Nakano et al., Proc. Natl. Acad. Sci. USA 79, 1593-1596, 1982; Franke et al., Mol. Cell. Biol. 1918-1924, 1985, Chakrabarti et al., Mol. Cell. Biol. 3403-3409, 1985, Fathi et al., Virology 97-105, 1986).

The VA prepared according to the present invention may also bear a marker gene. Such marker genes facilitate the isolation of the recombinant virus by means of techniques known per se. Such marker genes are known per se, and they include genes, which encode proteins, such as β-galactosidase, neomycin, alcohol dehydrogenase, luciferase, puromycin, hypoxanthine phosphoribosyl transferase (HPRT), hygromycin, secreted alkaline phosphatase or green and blue fluorescence proteins.

It is understood, that also other methods for the preparation of the recombinant virus of the present invention are possible, for example the cloning method described in the following example in more detail.

### Growth and Purification of the Viruses

### Growth of the MVA Virus

The MVA virus is a highly attenuated vaccinia virus derived from vaccinia virus strain Ankara (CVA) by serial long time passages on primary chicken embryo fibroblast (CEF) cultures. For a general overview over the history of the production, the characteristics and the use of the MVA strain reference may be made to the summary, which has been published by Mayr et al. in infection 3, 6-14 [1975]. Due to the attenuation in CEF, the MVA virus replicates with high titers in this avian host cell. However, in mammalian cells the growth of MVA is strongly inhibited, and a typical plaque formation is not detectable. Thus, the MVA virus was grown on CEF cells. For the preparation of CEF cells 11 days old embryos from incubated chicken eggs were isolated, the extremities were removed and the embryos were homogenized and dissociated in a solution composed of 0.25% Trypsin, for 20 min at 37° C. The resulting cell suspension was filtrated, and the cells were sedimented by centrifugation at 2000 rpm in a Sorvall RC-3B centrifuge for 5 min at room temperature resuspended in 10 volumes of Medium A (MEM Eagle, e.g. available from Life Technologies GmbH, Eggenstein Germany) and again sedimented by centrifugation at 2000 rpm in a Sorvall RC-3B centrifuge at room temperature. The cell pellet was again suspended in medium A containing 10% fetal calf serum (FCS), penicillin (100 units/ml), streptomycin (100 mg/ml) and 2 mM glutamine, so that a cell suspension was formed which contained 500000 cells/ml. CEF cells obtained in this manner were distributed on cell culture dishes. These were allowed to grow according to the desired cell density in medium A in a CO₂ Incubator for 1-2 days at 37° C., and were used either directly or after a further cell passage for infection. A detailed description for the preparation of primary cultures may be found in the book of R. I. Freshney, “Culture of animal Cell”, Alan R. Liss Verlag, New York [1983] chapter 11, S. 99 ff.

MVA viruses were used for infection as follows. The CEF cells were grown in 175 cm² cell culture flasks. At 90 to 100% confluence, the medium was removed and the cells were incubated for 1 hour with a MVA virus suspension (0.01 infectious units (IU) per cell, 0.02 ml/cm²) in Medium A. Then, more medium A was added (0.2 ml/cm²), and the flasks were incubated 2 to 3 days at 37° C. (until approx 90% of the cells exhibited cytopathogenic effect). Raw virus strain preparations were prepared by scraping the cell monolayers into the medium and by pelleting the cell material by centrifugation at 3000 rpm in a Sorvall RC-3B centrifuge for 5 min at 4° C. The raw virus preparation was stored at −20° C. prior to further processing (e.g. virus purification).

### Virus Purification

The purification steps done to obtain a virus preparation which was as pure as possible and which contained no components specific for the host cell resembled those described by Joklik (Virology 18, 9-18 [1962]. Raw virus strain preparations which have been stored at −20° C. were thawed and suspended once in PBS (10 to 20× of the sediment volume), and the suspension was centrifuged as above. The new sediment was suspended in 10× volume Tris buffer 1 (10 mM Tris HCl, pH 9.0) and the suspension was treated with ultrasound for a short period (Labsonic, L. B. Braun Biotech International, Melsungen, Germany; 2×10 sec at 60 Watt and room temperature) to further diminish the cell debris and to release the virus particles from the cellular material. The nuclei and larger debris were removed in the following short centrifugation of the suspension (Sorvall GSA rotor, available from DuPont Co., D-6353 Bad Nauheim, Germany, 3 min at 3000 rpm and 10° C.). Again, the sediment was suspended once in Tris buffer 1, treated with ultrasound and centrifuged as described above. The collected supernatants which are free of virus particles were combined and layered over a pad of 10 ml 36% sucrose in 10 mM Tris HCl, pH 9.0 and centrifuged in a Beckman SW 27/28 rotor for 80 min at 13500 rpm at 4° C. The supernatant was discarded, and the sediment containing the virus particles was taken up in 10 ml of 1 mM Tris HCl, pH 9.0, homogenized by a short treatment with ultrasound (2×10 sec at room temperature, device as described above) and applied to a 20-40% sucrose gradient (sucrose in 1 mM Tris HCl, pH 9.0) for further purification. The gradient was centrifuged for 50 min in a Beckman SW41 rotor at 13000 rpm and 4° C. Following centrifugation separate bands containing the virus particles were harvested by pipetting after aspiration of the volume over the band. The sucrose solution obtained was diluted with three volumes of PBS, and the virus particles were again sedimented by centrifugation (Beckman SW27/28, 60 min at 13500 rpm, 4° C.). The pellet now primarily composed of pure virus particles was resuspended in PBS and equilibrated to a virus concentration corresponding to an average of 1 to 5×10⁹ IU/ml. The purified virus stem solution was stored at −80° C. and either used directly or diluted with PBS for the following experiments.

The modified vaccinia virus Ankara (MVA), a highly attenuated vaccinia virus strain with limited host spectrum is not able to propagate in cells derived from humans and in most other mammalian cell lines studied. However, since viral gene expression is not affected in non-permissive cells, the recombinant MVA may be used as an unusually safe and efficient expression vector and recombinant vaccine.

Thus, in one embodiment of the present invention MVA viruses were constructed which enable the expression of the structural HCV antigen under the control of the vaccinia virus early/late promoter P7.5.

The recombinant MVA virus expressing a structural HCV antigen, may e.g. be used for the immunization for immune therapy in cancer patients.

Furthermore, the recombinant MVA virus expressing structural HCV antigens may be used in combination with antigen presenting cells, e.g. dendritic cells, macrophages and B cells, for the immunization of humans for immune therapy.

Furthermore, the recombinant MVA virus may be used in combination with antigen presenting cells, e.g. dendritic cells, ex vivo for the formation of immune effector cells which may be applied to humans for immune therapy.

The recombinant MVA virus may further be used for the preparation of recombinant structural HCV proteins.

The MVA vaccinia viruses of the invention will be transformed for the preparation of vaccines or therapeutics into a pharmaceutically acceptable form. Here, one can profit from the experience made with the preparation of MVA vaccines used for vaccination against pox (as described by Stickl, H. et al. [1974] Dtsch. med. Wschr. 99, 2386-2392). Usually about 10⁶ to 10⁸ recombinant MVA particles in 100 ml phosphate buffered saline (PBS) were freeze dried in the presence of 2% peptone and 1% human albumin in an ampoule, preferably a glass ampoule. The lyophilisate may contain an extending agent (such as mannitol, dextran, sugar, glycine, lactose or polyvinyl pyrrolidone) or other adjuvants (such as antioxidants, stabilizers etc.), which are suitable for parenteral administration. Then, the glass ampoule is closed and may than be stored preferably at temperatures of less than −20° C. for several months.

For vaccination or therapy the lyophilisate may be solved in 0.1 to 0.5 ml of an aqueous solution, preferably physiological salt solution and administered either parenterally, e.g. by intramuscular vaccination, or locally, e.g. by intradermal vaccination or by vaccination into a tumor or to a site of a tumor. Vaccines or therapeutics of the present invention will preferably be injected in an intramuscular manner (Mayr, A. et al. [1978] Zbl. Bakt. Hyg., I. Abt. Orig. B 167, 375-390). The route of administration, the dose and the number of administrations may be optimized by the person skilled in the art in a known manner. It may be suitable to administer the vaccine over a longer period of time to obtain suitable immune responses against the foreign antigen.

The invention also comprises eukaryotic cells infected with the recombinant MVA viruses. These include e.g. chicken embryo fibroblast cells, baby hamster kidney cells, preferably BHK-21 cells or antigen presenting cells such as e.g. dendritic cells, macrophages or B lymphocytes.

The recombinant MVA viruses are employed as described above in more detail for the preparation of a vaccine, which may be used for therapy and prophylaxis of HCV infections and diseases caused thereby, in particular chronical liver diseases and liver tumors. Naturally, by means of the recombinant virus also the recombinant HCV structural proteins may be produced and obtained following isolation and purification in pure form. According to the present invention, recombinant HCV proteins are meant to be structural HCV antigens, wherein said term also includes functional parts of the structural HCV antigens or epitopes, in particular surface epitopes of the structural HCV antigens.

The invention also includes DNA or RNA of the recombinant MVA.

In the following, the invention is described in more detail with reference to the examples. However, the invention is not limited to said particular embodiment, but may be modified in the scope of the description in combination with the accompanying claims.

### BRIEF DESCRIPTION OF THE DRAWINGS

The accompanying figures also serve as a further understanding of the invention, in particular of the examples. In the Figures there is shown:

FIG. 1

Construction and genomic structure of recombinant MVA containing gene sequences for HCV structural proteins. (A) A schematic map of the MVA genome for the restriction endonuclease HindIII. The HCV encoding sequences were under the transcriptional control of the vaccinia virus early/late promoter P7.5 and were inserted at the site of deletion III within the MVA genome by homologous recombination. Flank 1 and flank 2 relate to MVA DNA sequences present nearby the deletion site III and serve to direct the recombination into the MVA genome. Rec2 shows the positions of the 283 bp repetitive MVA DNA sequences corresponding to the right end of flank 1 and enabling the removal of the K1L selectable marker from the genome of the finished recombinant viruses by homologous recombination. The illustrations of the genome structures of the recombinant MVA-HCV-151-661, MVA-HCV-1-661 and MVA-HCV-1-742 are shown below. (B) PCR analysis of viral DNA for the control of gene sequences inserted at the deletion site III. Genomic DNA of wild type MVA and recombinant MVA-HCV-151-661, MVA-HCV-1-661 and MVA-HCV-1-742 served as a matrix DNA for the amplification of characteristic DNA fragments, separated by agarose gel electrophoresis. The 1 kB ladder (Gibco) was given as a marker (M) for molecular weight in base pairs (bp).

FIG. 2

Synthesis of HCV core and E2 proteins by recombinant MVA. BHK-21 cells were infected with 10 IU/cell MVA or MVA-HCV and harvested after 24 h (A, B) or at the time points indicated (C, D) after infection. Proteins from cell lysates were separated by SDS 10% PAGE and analyzed by western blotting by means of monoclonal mouse anti HCV core (A, C) or E2 specific polyclonal rabbit antibody (B, D). HCV specific protein bands are marked by arrow heads. The positions and molecular masses (in kDa) of the protein standards are shown in lanes MW.

FIG. 3

Glycosylation by cell associated and secreted E2 proteins prepared by recombinant MVA. Total Cell proteins (A) or protein precipitates of supernatants (B) from MVA infected BHK-21-cells were denaturated in SDS/β-mercaptoethanol, incubated in the presence (+) or absence (−) of endoglycosidase PNGase F, separated on SDS-10% PAGE and analyzed by western blotting with polyclonal rabbit anti E2 serum. HCV-E2 specific protein bands are marked by arrow heads. The numbers in lane MW relate to the positions and molecular weights (kDa) of the protein standards.

FIG. 4

Analysis of the EndoH sensitivity by cell associated and secreted E2 proteins prepared by recombinant MVA. BHK-21-cells were infected for 24 h with recombinant MVA/151-661 or MVA-HCV/1-661 or non-recombinant MVA at a MOI of 10. Total cell proteins (A) or protein precipitates of the supernatants (B) from infected cultures were denatured, incubated in the presence (+) or absence (−) of PNGase F or EndoH, separated on SDS-10% PAGE and analyzed by western blotting with polyclonal rabbit anti E2 serum. HCV-E2-specific protein bands are marked by arrow heads. The numbers in lane MW relate to the positions and molecular weights (kDa) of the protein standards.

FIG. 5

Specific lysis of E1 peptide pulsed target cells by CTL from MVA-HCV vaccinated BALB/c mice. Splenocytes were obtained from mice, which had been vaccinated with recombinant MVA-HCV/151-661 (A), MVA-HCV/1-661 (B) or MVA-HCV/1-742 (C) and restimulated in vitro with E1 peptide as 312-326. These effectors were tested for cytotoxicity in the given E:T relationship in a 4 hours ⁵¹Cr release test against P815 target which had been pulsed with E1 peptide as 312-326 (▪) or Flu peptide (▴). Splenocytes from mice which were vaccinated with non-recombinant MVA served as a control and were tested for P815 targets which had been incubated with E1 peptide as 312-326 (♦) or Flu peptide (X).

FIG. 6

E2 specific antibody response in BALB/c mice, which were vaccinated with recombinant MVA. Twofold dilution series of sera from BALB/c mice which had been immunized for three times with 10⁸ IU of recombinant or wild type MVA were tested by means of ELISA for E2 specific antibodies. The log(2) values of the normalized averaged anti E2 titers of all seroconverted animals are shown. The standard deviations are expressed by bars. The numbers at the top show the relationships between the seroconverted animals and the totally analyzed animals.

### DETAILED DESCRIPTION

### Materials and Methods

### Cell Lines and Viruses

Primary chicken embryo fibroblasts (CEF), Baby hamster kidney BHK-21

(ATCC CCL-10), rabbit kidney-RK-13 (ATCC CCL-37) and mouse P815 (H-2d, ATCC TIB64) cells were grown in minimal essential medium (MEM) or RPMI 1640 medium supplemented with 10% fetal calf serum (FCS). The cells were maintained in a humid air 5% CO₂ atmosphere at 37° C.

Vaccinia virus MVA (cloned isolate F6, 45, 60) was routinely propagated and its titer was detected by a vaccinia virus specific immune staining in CEF to determine the infectious Units (IU)/ml. Virus from the 582^{nd} CEF run were used for this study. Recombinant virus MVA-LZ encoding the E. coli lacZ reporter gene (60) was used in the plaque reduction assay to determine vaccinia virus MVA specific neutralizing antibodies.

### Plasmid Constructs

For the construction of MVA vector plasmids containing structural HCV genes, DNA fragments from cloned HCV-cDNA were prepared which originally were obtained from Y. Wang (University of Beijing, GENBANK® Accession number D10934, 63, 38). A DNA segment containing the coding sequence for the HCV amino acids (aa) 1 to 742 was obtained by EcoRI and HindIII cleavage of pUC18/CE1E2, treated with Klenow polymerase, for the formation of blunt ends and cloned into the single Pmel-site of pIIIdHR-P7.5, to obtain pIIIdHR-P7.5-HCV (1-742).

To obtain MVA vector plasmids pIIIdHR-p7.5-HCV (1-661) and pIIIdHR-P7.5-HCV (151-661), DNA fragments encoding HCV-aa 1-661 or as 151-651 were amplified by means of a polymerase chain reaction (PCR) from the same HCV-cDNA matrix, wherein the following oligonucleotides were used: HCV-5′-1 5′-CAT GGG AAT TCC CAT GAG-3′, HCV-5′-151 5′-GGC GCT GCG AAT TCC ATG GCG CAT GGC GTC CGG-3′ and HCV-3′-661 5′-GGG GGG GAA TTC TCA CTC TGA TCT ATC CCT GTC-3′ (Eco RI-sites are underlined). The PCR products were cleaved with EcoRI, blunt ended with Klenow and cloned into the PMEI site of pIIIdHR-P7.5.

### Formation of Recombinant Viruses

Monolayers of nearly confluent BHK-21 cells in tissue culture plates with six wells (Corning, Corning, N.Y., USA) were infected with MVA at a multiplicity of 0.01 infectious units per cell and transfected 90 min after infection with 1.5 μg plasmid DNA per well by means of FuGENE 6-transfection reagent (Roche Molecular Biochemicals Mannheim, Germany) according to the manufacturer's specifications. 24 h after infection the cells were harvested and processed as described for the isolation of rMVA by selection for transient extension of the host cell spectrum (58). Recombinant viruses were seen after the formation of typical infection foci in RK-13 cell monolayers. The RK-13 cells did not support the productive growth of parenteral MVA. After 3 propagation cycles of plaque purification in RK-13 cells, the rMVA were sent through BHK-21 cells to remove the selectable marker gene K1 L. Virus starting materials were prepared in BHK-21 cells, purified via a two-step ultra centrifugation through a sucrose pad, the titre was determined on CEF-cells by means of vaccinia virus specific immune staining, separated into aliquots and stored at −80° C.

### Analysis of Recombinant Proteins

Monolayers of BHK-21 cells were infected with 10 infectious rMVA units per cells. After 24 h the infected cells were harvested, collected by centrifugation and lysed in SDS gel loading buffer (50 mM Tris-HCl, pH 6.8, 2% SDS, 0.04% bromophenolblue, 84 mM β-mercaptoethanol, 20% glycerol). Total cell proteins were electrophoresed on a SDS polyacrylamide gel and electroblotted onto nitrocellulose. The blot membranes were blocked for 1 h in phosphate buffered saline buffer (PBS) with 2% (W/V) bovine serum albumin (BSA) and 0.05% (V/V) Tween 20, then incubated for 4 h with polyclonal rabbit anti-HCV-E2 antibodies (serum RE2116, 1:5000), polyclonal rabbit anti-HCV-antibodies (Antigenix, Amerika Inc./BioTrade, Vienna, Austria, 1:10000) or monoclonal mouse anti-HCV-core-antibodies (1:5000, kindly offered by Ramsey C. Cheung, Stanford University School of Medicine, Stanford, Calif., USA), diluted in the same PBS buffer. After washing with 0.05% Tween 20 in PBS, the blots were incubated for 1 h with anti-rabbit or anti-mouse IgG-antibody, conjugated to horseradish peroxidase (Dianova, Hamburg, Germany) and diluted 10000 fold in blocking buffer, incubated, again washed and developed by means of Lumi-Light western blotting substrate (Roche Molecular Biochemicals, Mannheim, Germany). For the control of secreted recombinant HCV proteins cell free supernatants from MVA infected cultures which were grown in serum free OPTI-MEM® (Gibco BRL), were collected 24 hours after infection. Proteins from the supernatants were precipitated with an equal volume of ice-cold ethanol, obtained by an 1 h centrifugation at 10000×g/4° C., resuspended in PBS and subjected to a western blot analysis.

The endoglycosidases EndoH and PNGaseF (New England Biolabs, Frankfurt am Main, Germany) were used for the deglycosylation of recombinant proteins. The total proteins precipitated from infected cells or from culture supernatants were denatured for 10 min at 100° C., 2 h at 37° C. in 0.5% SDS, 1%β-mercaptoethanol, with EndoH or PNGase F, as described by the manufacturer's instructions, cleaved and analyzed by Western blotting.

### Animal Immunization and Obtaining Serum Samples

Groups of five BALB/c mice (6 to 8 weeks old, obtained by Charles River, Sulzfeld, Germany) were intraperitoneally immunized with 1×10⁸ infectious MVA or rMVA units (in 0.5 ml sterile PBS) per animal at the days 0, 38 und 81. The mice were bled through the retroorbital-plexus at days −7, 21, 48, 103 and 158. The blood was collected in a microcentrifuge tube, incubated for 4 h at room temperature and centrifuged for 10 min at 2700×g/4° C. The sera obtained were stored at −20° C.

### Antibody Measurement by ELISA

Enzyme immunosorbent assays (ELISA) were used for the determination of the presence of antibodies against HCV nucleus und E2-Antigens in serum probes. The antigens used for coating the 96 well flat bottom plates (MaxiSorp Surface, Nunc, Wiesbaden, Germany) in a concentration of 1 μg/ml were HCV nucleus protein (aa 2 to 192, Bio Trade, Vienna, Austria) or HCV E2 produced in E. coli (aa 450 to 565). The antigens were suspended in PBS with 0.02% sodium azide, plated with 50 μl/well and incubated over night at 4° C. Subsequently, the content of each well was removed and washed three times with PBS plus 0.05% Tween 20 (PBS-T). Blocking buffer (1% BSA in PBS-T) was added at 200 μl/well, and the plates were incubated for 1 h at 37° C. The plates were washed with PBS-T, and twofold serial dilutions of the serum samples in blocking buffer were added in a volume of 100 μl/well and incubated for 3 h at 37° C. After three washing steps with PBS-T, alkaline phosphatase conjugated goat anti mouse immunoglobulin G (Dianova, Hamburg, Deutschland, 1000× diluted in blocking buffer) was added and incubated for 1 h at 37° C. After 8 times washing of the plates with PBS-T, the wells were developed with para-nitrophenylphosphate substrate (Sigma, Deisenhofen, Germany). After 30 min incubation at room temperature the reaction was stopped by the addition of 0.5 M NaOH, and the extinction was measured at 405 nm with a microplate reading device (Model 550, Bio Rad Laboratories, Munich, Germany). The antibody titers were calculated as a twofold serial dilution resulting in an optical density (OD), which was twice as high as the Cutoff. The cutoff value was estimated as the average OD of sera from control mice, which were vaccinates with non-recombinant MVA.

### Analysis of MVA Specific Antibody Answers

Vaccinia virus MVA specific antibodies were analyzed by a plaque reduction test by means of recombinant MVA-LZ. Twofold serial dilutions of sera from immunized mice were reacted with 200 infectious MVA-LZ units in a total volume of 200 μl of PBS and incubated for 3 h at 37° C. Thereafter, the confluent BHK-21 monolayer were incubated in a double approach, and foci of virus infected cells were visualized 48 h after vaccination by staining with 5-Bromo-4-chloro-3-indolyl-β-galactopyranoside substrate (X-Gal, Roche Molecular Biochemicals, Mannheim, Germany) as described above (16). The blue colored foci were counted, and the number obtained with each serum was compared to controls with preimmune sera or without mouse serum. The antibodies were calculated as twofold serial dilution, which resulted in a 50% reduction of the foci number.

### Splenocyte Cultures and Cytotoxicity Test

The methods were done essentially as described before (5). Splenocytes, obtained 12 weeks after the last virus vaccination of the mice, were used as in vitro responder cells. 5×10⁶ cells were grown in the presence of 5 μg/ml of E1 peptide (HCV as 312-326) in RPMI1640 medium supplemented with 10% FCS, 25 μM 2-mercaptoethanol, 1 mM sodium pyruvate and 2 mM L-glutamine. On the second day, 10% TCGF was added to the cultures. After 7 days the effector cells were harvested and tested for specific cytotoxicity, wherein a ⁵¹Cr release test was used. 1×10⁶ P815 or T2 target cells were incubated with 75 μCi Na⁵¹ CrO₄ in 200 μl RPMI/10% FCS 1 h at 37° C. The cells were washed three times with RPMI/10% FCS and incubated three times with 5 μg/ml E1 peptide (HCV as 312-326) or 1 μg/ml A/PR/8/34-influenza virus matrix protein M1 peptide for 30 min at 37° C. Responder and target cells were co-cultivated in different ratios in plates with 96 wells for 5 h at 37° C. 30 μl supernatant were harvested to determine the amount of released ⁵¹Cr. All samples were counted in a TopCount NXT (Packard, Downers Grove Ill.), and the percentage of specific release was calculated as:

[(experimental release−spontaneous release)/(total release−spontaneous release)]×100. The release was measured in counts per min. The tests were carried out in a double approach. For restimulation; 5×10⁶ irradiated splenocytes (3000 rad) which were derived from non-immunized BALB/c mice and which were incubated for 2 h with 10 μg/ml E1 peptide (HCV as 312-326) were added to 1×10⁶ effector cells and grown for 7 days in RPMI medium supplemented with 10% FCS, 25 μM 2-mercaptoethanol, 1 mM sodium pyruvate, 2 mM L-glutamine and 10% TCGF.

### Results

Design and Isolation of rMVA for the Expression of HCV-C-E1-E2 Genes

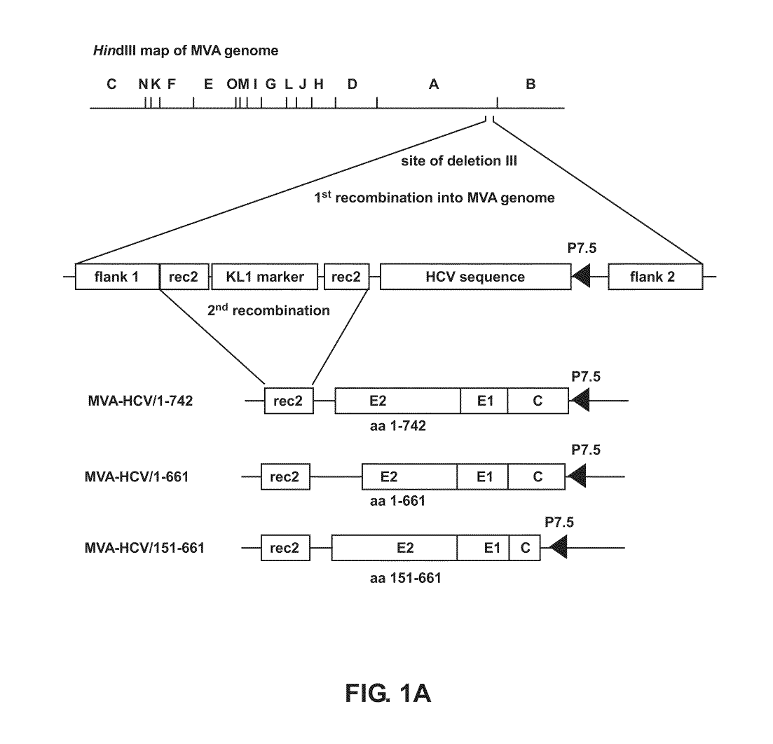

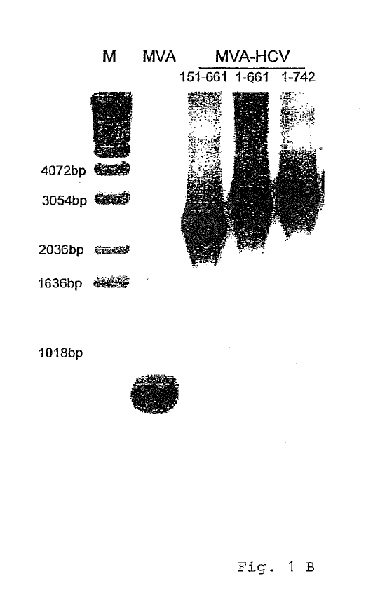

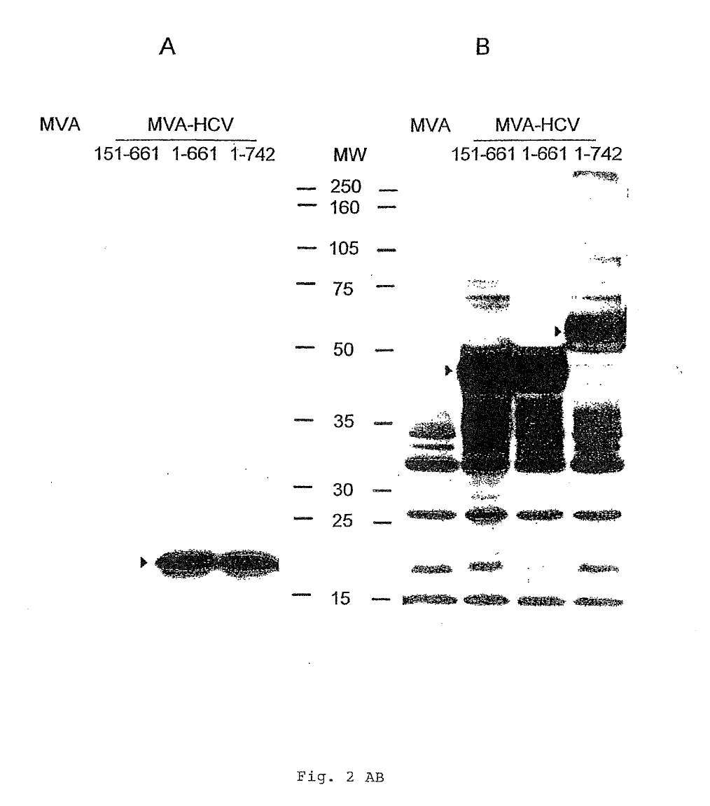

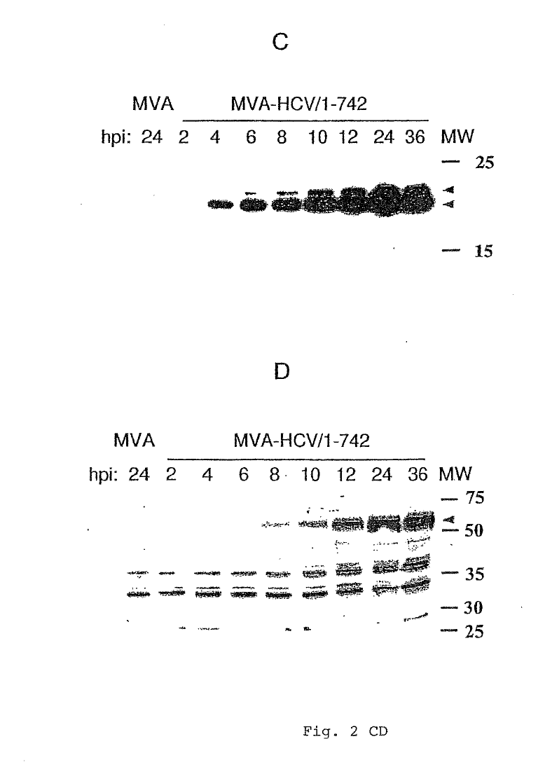

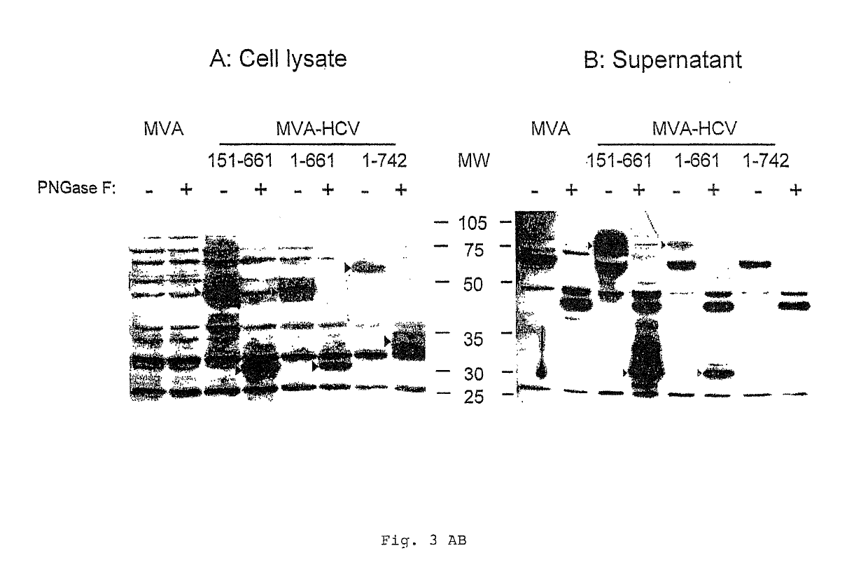

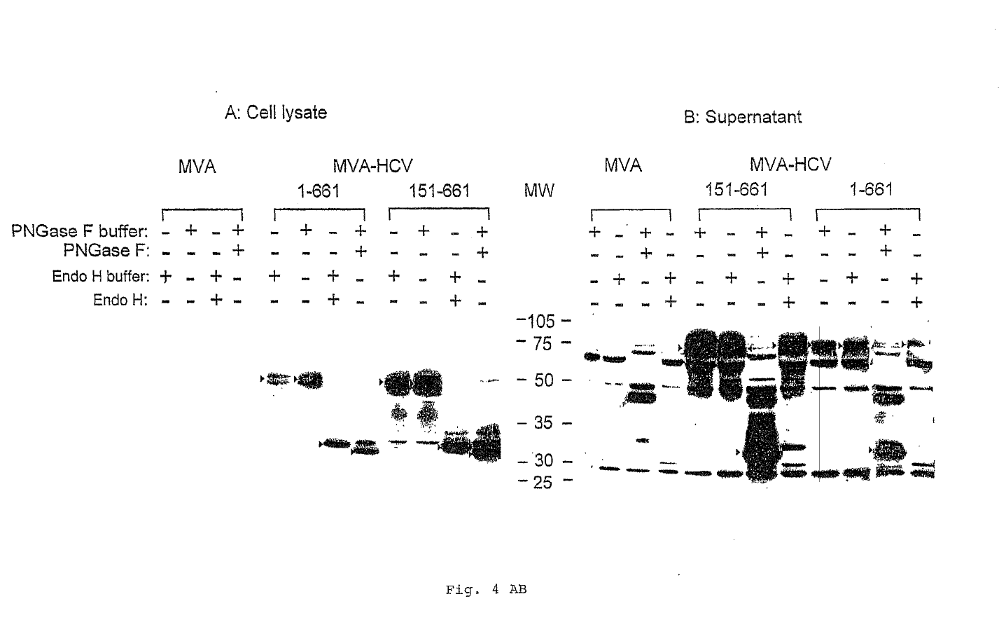

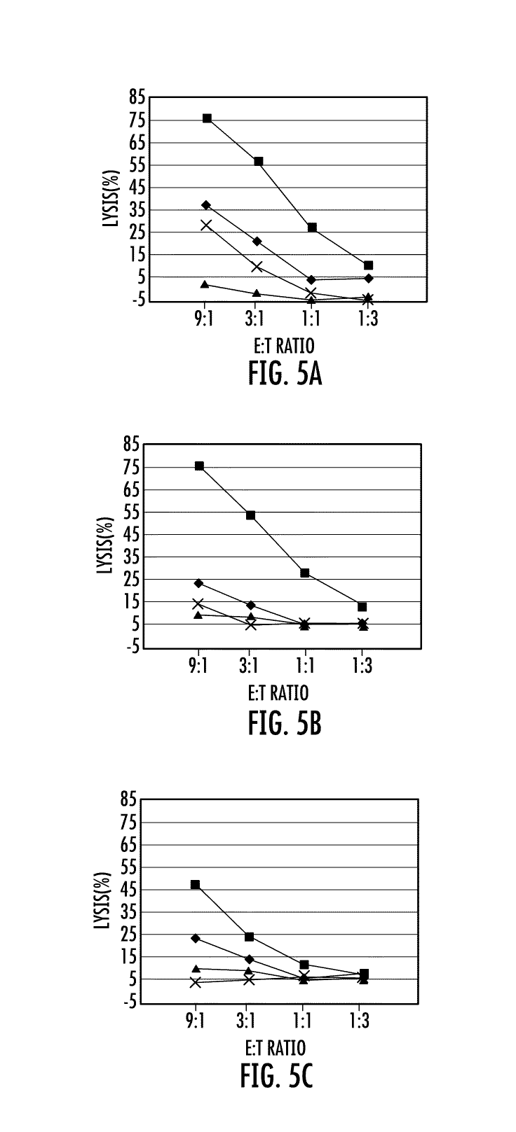

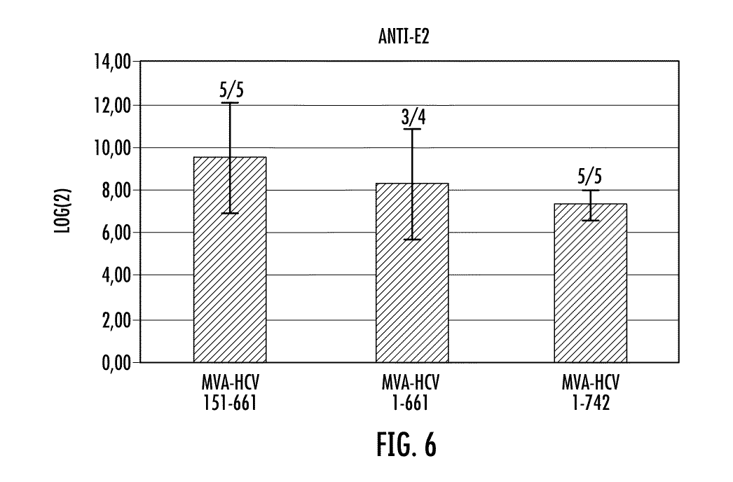

Prior investigations showed a suitable formation and isolation of rMVA by means of vector plasmids which enables a host range selection on the basis of a transient expression of the vaccinia virus K1L gene (58). For the use of said novel technique the HCV target gene sequences were cloned into the MVA vector plasmid pIIIdHR-P7.5. The resulting plasmids pIIIdHR-HCV/1-742, pIIIdHR-HCV/1-661 und pIIIdHR-HCV/151-661 contained HCV-cDNA encoding amino acids 1 to 742, 1 to 661 or 151 to 651 of the polyprotein. rMVA was formed in BHK-21 cells which have been infected with MVA, and which were transfected with pIIIdHR-HCV/1-742, pIIIdHR-HCV/1-661 or pIIIdHR-HCV/151-661. Dilution series of the infected cell lysates were plated to RK-13 cells which enabled a selective growth of recombinant viruses at transient expression of the selectable marker gene K1L. After 3 cloning passages on RK-13 cells only rMVA was detectable, which was confirmed by PCR analysis of viral DNA (data not shown), and the virus isolates were further plaque purified in BHK 21 cells. The growth of rMVA in said cells was independent of K1L gene expression and resulted to the loss of the marker gene by homologous recombination between repetitive DNA sequences which flanked the K1L gene within the viral genomes (FIG. 1A). After several passages through BHK-21 cells, the PCR analysis of viral DNA revealed that the viral genome contained stably integrated HCV target gene sequences but no K1L genes any longer (FIG. 1B). Amplification and purification of the obtained viruses MVA-HCV/1-742, MVA-HCV/1-661 and MVA-HCV/151-661 resulted in virus preparation with high titers which were used for further analysis.

Synthesis of HCV Proteins after rMVA Infection

Since the formed rMVA should be tested as a vector vaccine candidate for the delivery and immunological characterization of HCV antigens, the production of HCV proteins in tissue culture infection should be thoroughly determined. All rMVA containing different coding sequences of the core, E1 and E2 proteins controlled the synthesis of the structural HCV proteins, as has been shown by SDS PAGE analysis and immunoblotting of BHK 21 cell lysates which were harvested 24 h after infection (FIG. 2A, B). Monoclonal antibodies raised against the HCV core protein, resulted in specifically equal amounts of an about 21 kDa core protein, prepared in MVA-HCV/1-742 or MVA-HCV/1-661 infected cells, whereas no such protein band could be detected after infection with MVA-HCV/151-661 or non-recombinant MVA (FIG. 2A). At later times and with increased amounts of core protein a second 23 kDa protein band could be detected. The synthesis of HCV envelope proteins was controlled with an E2 specific polyclonal rabbit antiserum. According to FIG. 2B, all three recombinant viruses formed E2 polypeptides which had been visualized as proteins with molecular weights of about 60 kDa for MVA-HCV/1-742 and about 50 kDa for MVA-HCV/1-661 and MVA-HCV/151-661. The latter viruses both contain expression cassettes for HCV polyproteins having E2 sequences truncated at the C-terminus. Several other smaller protein bands were also stained by the polyclonal serum and possibly represent proteins which are not HCV encoded, since they have been also detected in control lysates from cells infected with wild type MVA.

Further, the kinetics of the HCV protein synthesis in rMVA infection should be controlled. We synchronously infected BHK-21 cell monolayers with 10 infectious units/cell of MVA-HCV/1-742 and prepared cell lysates for western blot analysis at several times during a period of 2 days after infection (FIGS. 2C, D). Already 4 h after infection the synthesis of HCV core protein could be detected FIG. 2C), whereas the first recombinant E2 protein (FIG. 2D) clearly occurred 8 h after infection. The amounts of both recombinant proteins clearly increased during a time period of 34 h after infection. Lysates of cells which had been infected for 24 h with wild type MVA served as negative controls.

Analysis of Cell Associated and Secreted Forms of E2 Antigens, Prepared by rMVA

Posttranslational modifications of the delivered antigens may substantially influence the immunogenicity and the protection capacity. The HCV envelope proteins E1 and E2 are highly modified by glycosylation and are possibly type I transmembrane glycoproteins with a carboxyterminal hydrophobic anchor domain. The removal of the E2 transmembrane anchor results in the secretion of the E2 ectodomain (overview c.f. 17). Upon infection of mammalian cells the eukaryotic MVA expression system should result in the occurrence of authentic posttranslational modifications of recombinant proteins. For the control of said processes the E2 proteins were biochemically characterized, which are synthesized during infection with rMVAs. BHK-21 cells were infected with MVA-HCV/1-742, MVA-HCV/1-661 and MVA-HCV/151-661. Total cell lysates (FIG. 3A) or precipitates of supernatants from infected cells (FIG. 3B) were treated with PNGase F and analyzed by western blotting by means of polyclonal anti-E2-antiserum. Expressed E2 proteins with ˜60 kDa (MVA-HCV/1-742) or ˜50 kDa (MVA-HCV/1-661 and MVA-HCV/151-661) in cell lysates were reduced to a size of about 33 kDa (full length E2) or 31 kDa (truncated E2), respectively, corresponding to the unglycosylated peptide backbone of E2 (FIG. 3A). Secreted E2 could only be detected after infection with the recombinant viruses MVA-HCV/1-661 and MVA-HCV/151-661, which produce a truncated E2 in the transmembrane domain. Secreted E2 is more strongly modified than cell associated E2 with an apparent molecular weight of ˜75 kDa. Upon treatment with PNGase F said proteins will also be reduced to a size of about 31 kDa, corresponding to a peptide backbone of the truncated E2. In the next step the glycosylation type in said secreted E2 proteins was further characterized. Cells were infected with MVA-HCV/1-661 and MVA-HCV/151-661, cell lysates or supernatants of infected cells were treated with endoglycosidase H (FIG. 4). However, intracellular E2 is sensitive against EndoH, as has been detected by a reduction of MW of ˜50 kDa to ˜32 kDa (FIG. 4A), but secreted E2 is not affected as far as possible (FIG. 4B). The E2 bands of the PNGase F cleavage had a lower MW than the corresponding bands of the Endo H cleavage, since the latter enzyme leaves a GlcNAc residue at the side chain of Asn after the cleavage. This observation corresponds to a more complex glycosylation of c-terminally truncated E2 during the passage through the Golgi apparatus.

Vaccination of Mice with rMVA Causes HCV and Vaccinia Virus Specific Immune Responses

Now, the immunogenicity of different rMVA vaccines was tested in mice, since it was known, that the production of the HCV core protein by vaccinia virus recombinants mediates an immunosuppression in mice (36). Additionally, the possible modulation of the immune responses by full length HCV core protein, raised by 2 of our rMVA vaccines, should be determined. Thus, HCV specific humoral or cellular responses were analyzed, which were directed against HCV-E1 and -E2 antigens. After three times of intraperitoneal immunization of BALB/c mice with recombinant or wild type MVA HCV-E1, specific CTL responses with an E1 (312-326)-BALB/c-epitope were investigated. Splenocytes which were derived from Animals immunized with MVA/HCV/151-661 or MVA-HCV/1-661, showed E1 specific lysis of 75% with an E:T ratio of 9:1, splenocytes of animals which were immunized with MVA-HCV/1-742 resulted in a specific lysis of 47%, when they were stimulated with the E1 peptide (FIG. 5). A higher E1 specific lysis in cultures with splenocytes of mice was observed which were immunized with rMVA expressing C-terminally truncated E2 proteins.

Serum samples of the same BALB/c mice were furthermore investigated for antibody responses against the HCV E2 protein by ELISA (FIG. 6). E2 specific antibodies with an average of the titers of 1:2512 (MVA-HCV/151-661), 1:933 (MVA-HCV/1-661) and 1:176 (MVA-HCV/1-742) were detected. Higher amounts of anti-E2 antibodies were found in animals which had been immunized with MVA-HCV/151-661, although the differences beyond the groups were statistically not significant.

The HCV core protein may exhibit as already mentioned an immunomodulatory function. Now, it was investigated whether the higher anti E2 antibody response of MVA-HCV/151-661-injected mice could be addressed to the large truncation of the core protein in said virus. It was expected, that the HCV nucleus also modulates the induction of the vaccinia virus MVA specific immune responses. Thus, the amounts of MVA neutralizing antibodies in the same mouse sera as were used for ELISA were measured. According to table 1, no difference of the anti MVA antibody response beyond the groups immunized with recombinant or wild type MVA expressing full length or truncated core protein was detected.

Summary of the Experiments

Prior experiments established data for the immunogenicity and protection effect of recombinant MVA vaccines delivering viral antigens in a series of animal model systems (61, 29, 27, 64, 65, 44, 59). However, there was still an urgent need for a vaccine against HCV infection and a vaccine candidate had to be identified which has a suitable immunogenic and possibly protective capacity. For the investigation of the suitability of MVA for the production of HCV antigens a series of rMVA with inserted expression cassettes of HCV gene sequences were constructed, which code for the structural proteins C-E1-E2. We wanted to determine the characteristics of recombinant MVA, which was designed to produce HCV-E1-E2 envelope proteins or secreted E2 antigen with or without full length HCV Capsid/Core protein. The formation and isolation of rMVA resided in the transient host range gene selection (58) and showed that this technique is particularly suited, when several MVA vector viruses have to be prepared for a comparative test. Recombinant MVA which expresses the different HCV encoding sequences under the control of the vaccinia virus early late promoter P7.5, could easily be obtained and initiating experiments showed, that all constructs produced similar amounts of structural HCV proteins in a corresponding manner.

The HCV core sequence is one of the most highly conserved regions in the viral genome reaching up to the viruses from different HCV genotypes and which renders the core protein interesting as an antigen for immunization (6, 57, 7). However, the inclusion of core into vaccine candidates is highly questionable, since its multifunctional characteristics are possibly involved in the regulation of the host cell apoptosis, transcription, transformation and immune presentation (43, 4). The expression of HCV sequences encoding polyprotein as 1-742 and 1-661 by recombinant MVA primarily led to the synthesis of the 21-kDa-core proteins (FIG. 2A). Only at later times of infection with increasing amounts of produced recombinant protein a second core-specific protein band at 23 kDa size was visible. The 23 kDa protein possibly is the non-mature core protein with 191 amino acids derived from an initial polyprotein cleavage, whereas the 21-kDa protein possibly is the mature core protein present in virus particles (28, 26, 51, 55, 66). Said data show an efficient early synthesis and processing of the core polypeptide in MVA-HCV vector infected cells.

After the infection with MVA-HCV/1-742 the synthesis of an approx. 60 kDa E2 protein was verified which strictly remained cell associated (FIG. 2B). The amplification of a 75 kDa C-terminally truncated E2 protein in cell culture supernatants suggested for the functionality of the secretory pathway in HCV infected cells. The comparison of cell-associated and secreted truncated E2, produced by MVA-HCV/1-661 and MVA-HCV/151-661, by SDS-PAGE (FIG. 3AB) showed, that the secreted form migrated more slowly. This occurred due to a different glycosylation. Accordingly both E2 proteins migrate after deglycosylation with PGNAse F on the same level. Furthermore, the secreted E2 was insensitive against a treatment with endoglycosidase H, which clearly shows the uptake of complex sugars during transport by the Golgi apparatus. However, the expression of the HCV-E1 protein was not analyzed formally, but the E2 protein forms a downstream portion of the HCV polyprotein and the demonstration of the E2 synthesis suggests for the production of the E1 protein sequences.

The correctness of this thought was verified by the detection of E1 specific T cell responses after vaccination with recombinant MVA-HCV viruses.

For the comparative test of the immunogenicity of the vaccines the BALB/c mice were vaccinated and investigated for the induction of immune responses, which were directly directed against HCV envelope proteins produced by all vector viruses. For the envelope antigens E1 and E2 specific CTL are present in the liver and in peripheral blood of HCV infected humans and chimpanzees (Walker 1996, Sem. Virol.). More recent data of the investigation of immune responses in HCV infected chimpanzees suggest that CD8+CTL are accompanied by an acute cessation of the infection (12). The analysis of the fine specificity of long durable CTL revealed 9 different peptide epitopes of the HCV proteins E1, E2, P7, NS2, NS3, NS5a, wherein 4 of said epitopes are present in E1/E2 sequences. The E1 epitope 312-326 was successfully used for in-vitro restimulation of splenocytes from vaccinated BALB/c mice, and epitope-specific CTL were detected which were induced by all 3 rMVA vaccines. Interestingly, the detection of E1 specific CTL was also possible more than 12 weeks after the last immunization. This suggests that immunizations over a long period could induce long lasting (memory phase) E1 specific T cell responses.

The co-synthesis of the HCV core protein did not affect the induction of E1 specific CTL responses, however, an immunization with vector viruses producing C-terminally truncated secreted E2 proteins, seemed to elicit higher E1 specific cytotoxic activities. The glycoproteins E1 and E2 form complexes, which in endoplasmic reticulum (ER) are retained at the C-termini of the proteins due to the interaction of hydrophobic transmembrane sequences (10). The removal or mutation of the E2 transmembrane region inhibits the formation of native E1-E2 complexes and prevents the correct folding of the E1 glycoprotein (11, 48). For a presentation by MHC-I-molecules the E1 antigen seems to be derived from the endoplasmic reticulum, but requires a cytoplasmic processing (54) and resides probably on a secondary export pathway for defect transmembrane glycoproteins into the cytosol as has been suggested for the influenza nucleoprotein or the cellular glycoprotein tyrosinase (2, 47). Due to this finding of the higher CTL activity after immunization with vector viruses producing secreted E2 one might speculate that the mobilization of truncated E2 for the secretory pathway makes available E1 preferably for the above processing pathway and enables an enhanced MHC-I restricted presentation of E1 antigens.

The protection achieved after the prophylactic vaccination with recombinant HCV-envelope antigens in the chimpanzee model was correlated with E2 specific antibodies (9). Accordingly, great attention was paid to the E2 protein as a vaccination antigen candidate for the induction of HCV neutralizing antibodies (68, 56). During the investigation for E2-specific humoral immune responses in the experiments specific antibodies were found which were formed after immunization in all 3 rMVA vaccines. Higher antibody titers were obtained in sera from animals vaccinates with vectors which synthesize secreted E2 proteins with truncated transmembrane domain. Naturally, the secretion of E2 could elicit better antibody responses, whereas the interaction of full length E1 and -E2 proteins for the formation of intracellular retained complexes could mask important epitopes and prevent an optimal immune presentation. The HCV core protein, produced by replication competent vaccinia virus, seems to suppress host immune responses, in particular to suppress the formation of vaccinia virus specific immune responses (Large et al., 1999, J. Immunol 162: 931-938). In the vaccination trials shown herein, corresponding to these data the best E2 specific antibody responses seemed to be induced by the MVA-HCV/151-661 vaccine, which produced truncated E2 in the absence of core antigen. The accompanying analysis of the vaccinia virus specific neutralizing antibody responses showed however, that nearly equal amounts of vaccinia virus specific immunity was elicited by all rMVA-HCV vaccines. However, the evaluation of the results of all immunization trials led to the conclusion that the HCV core antigen (at least in the present experiments) with replication deficient vaccinia virus MVA as expression vector had no detectable effect.

In summary, this investigation proves that rMVA may be used for an efficient production of mature core and envelope proteins. Moreover, the vaccination of BALB/c mice with rMVA vaccines induced comparable amounts of E2 specific antibodies and long lasting E1 specific cytotoxic T cell responses and evidences the usability of said viral vector for the further evaluation as a promising vaccine against HCV infection.

Thus according to the present invention rMVA was investigated as a potential vaccine candidate against a HCV infection. In the first step MVA vector vaccines were constructed which express all sequences encoding the main HCV virion components, to characterize the synthesis of recombinant HCV proteins upon MVA infection and to evaluate their immunogenicity in the use as vaccines.

In this investigation, a strong synthesis and an efficient posttranslational maturation of full length HCV structural proteins was detected, which were prepared upon in-vitro infection with rMVA. An induction of HCV-E1 and E2-antigen specific humoral or cellular immune responses in BALB/c mice for rMVA immunization could be shown. All vaccines caused balanced amounts of vaccinia virus specific circulating antibodies. The present data suggest for a high immunogenicity of rMVA expressed HCV structural antigens, including the complete HCV core antigen.

TABLE 1
Vaccinia virus neutralizing antibodies, induced by MVA vaccines

| | titer^{b} (reciprocal average log2)^{c} | | | |
|---|---|---|---|---|
| vaccine^{a} | d0 | d28 | d55 | d110 |
| MVA | <5.3 | 10.3 | 12.3 | 12.3 |
| MVA-HCV/151-661 | <5.3 | 9.5 | 12.3 | 12.3 |
| MVA-HCV/1-661 | <5.3 | 10.1 | 11.9 | 12.1 |
| MVA-HCV/1-742 | <5.3 | 9.9 | 12.3 | 12.3 |

^{a}10⁸ IU MVA vaccine, given on the intraperitoneal way at the days 0, 38, 81
^{b}Serum antibody titers were determined by the 50% vaccinia virus plaque reduction test
^{c}n = 5, maximum standard deviation is

### LITERATURE

1. Amara, R. R., F. Villinger, J. D. Altman, S. L. Lydy, O. N. SP, S. I. Staprans, D. C. Montefiori, Y. Xu, J. G. Herndon, L. S. Wyatt, M. A. Candido, N. L. Kozyr, P. L. Earl, J. M. Smith, H. L. Ma, B. D. Grimm, M. L. Hulsey, J. Miller, H. M. McClure, J. M. McNicholl, B. Moss, and H. L. Robinson 2001. Control of a Mucosal Challenge and Prevention of AIDS by a Multiprotein DNA/MVA Vaccine Science. 292:69-74.
2. Bacik, I., H. L. Snyder, L. C. Anton, G. Russ, W. Chen, J. R. Bennink, L.
Urge, L. Otvos, B. Dudkowska, L. Eisenlohr, and J. W. Yewdell 1997. Introduction of a glycosylation site into a secreted protein provides evidence for an alternative antigen processing pathway: transport of precursors of major histocompatibility complex class I-restricted peptides from the endoplasmic reticulum to the cytosol J Exp Med. 186:479-87.
3. Belyakov, I. M., L. S. Wyatt, J. D. Ahlers, P. Earl, C. D. Pendleton, B. L. Kelsall, W. Strober, B. Moss, and J. A. Berzofsky 1998. Induction of a mucosal cytotoxic T-lymphocyte response by intrarectal immunization with a replication-deficient recombinant vaccinia virus expressing human immunodeficiency virus 89.6 envelope protein J Virol. 72:8264-72.
4. Bergqvist, A., and C. M. Rice 2001. Transcriptional activation of the interleukin-2 promoter by hepatitis C virus core protein J Virol. 75:772-81.
5. Bruna-Romero, O., J. J. Lasarte, G. Wilkinson, K. Grace, B. Clarke, F. Borras-Cuesta, and J. Prieto 1997. Induction of cytotoxic T-cell response against hepatitis C virus structural antigens using a defective recombinant adenovirus Hepatology. 25:470-7.
6. Bukh, J., R. H. Purcell, and R. H. Miller 1994. Sequence analysis of the core gene of 14 hepatitis C virus genotypes Proc Natl Acad Sci USA. 91: 8239-43.
7. Cerny, A., J. G. McHutchison, C. Pasquinelli, M. E. Brown, M. A. Brothers, B. Grabscheid, P. Fowler, M. Houghton, and F. V. Chisari 1995. Cytotoxic T lymphocyte response to hepatitis C virus-derived peptides containing the HLA A2.1 binding motif J Clin Invest. 95:52130.
8. Choo, Q. L., G. Kuo, A. J. Weiner, L. R. Overby, D. W. Bradley, and M. Houghton 1989. Isolation of a cDNA clone derived from a blood-borne non-A, non-B viral hepatitis genome Science. 244:359-62.
9. Choo, Q. L., G. Kuo, R. Ralston, A. Weiner, D. Chien, G. Van Nest, J. Han, K. Berger, K. Thudium, C. Kuo, and et al. 1994. Vaccination of chimpanzees against infection by the hepatitis C virus Proc Natl Acad Sci USA. 91:1294-8.
10. Cocquerel, L., J. C. Meunier, A. Pillez, C. Wychowski, and J. Dubuisson 1998. A retention signal necessary and sufficient for endoplasmic reticulum localization maps to the transmembrane domain of hepatitis C virus glycoprotein E2 J Virol. 72:2183-91.
11. Cocquerel, L., C. Wychowski, F. Minner, F. Penin, and J. Dubuisson 2000. Charged residues in the transmembrane domains of hepatitis C virus glycoproteins play a major role in the processing, subcellular localization, and assembly of these envelope proteins J Virol. 74:3623-33.
12. Cooper, S., A. L. Erickson, E. J. Adams, J. Kansopon, A. J. Weiner, D. Y. Chien, M. Houghton, P. Parham, and C. M. Walker 1999. Analysis of a successful immune response against hepatitis C virus Immunity. 10:439-49.
13. Davis, G. L., D. R. Nelson, and G. R. Reyes 1999. Future options for the management of hepatitis C Semin Liver Dis. 19: 103-12.
14. Dietzschold, B., H. H. Wang, C. E. Rupprecht, E. Cells, M. Tollis, H. Ertl, E. Heber-Katz, and H. Koprowski 1987. Induction of protective immunity against rabies by immunization with rabies virus ribonucleoprotein Proc Natl Acad Sci USA. 84:9165-9.
15. Drexler, I., E. Antunes, M. Schmitz, T. Wolfel, C. Huber, V. Erfle, P. Rieber, M. Theobald, and G. Sutter 1999. Modified vaccinia virus Ankara for delivery of human tyrosinase as melanoma-associated antigen: induction of tyrosinase- and melanoma-specific human leukocyte antigen A*0201-restricted cytotoxic T cells in vitro and in vivo Cancer Res. 59:4955-63.
16. Drexler, I., K. Heller, B. Wahren, V. Erfle, and G. Sutter 1998. Highly attenuated modified vaccinia virus Ankara replicates in baby hamster kidney cells, a potential host for virus propagation, but not in various human transformed and primary cells J Gen Virol. 79:347-52.
17. Dubuisson, J. 2000. Folding, assembly and subcellular localization of hepatitis C virus glycoproteins Curr Top Microbiollmmunol. 242:135-48.
18. Dubuisson, J., H. H. Hsu, R. C. Cheung, H. B. Greenberg, D. G. Russell, and C. M. Rice 1994. Formation and intracellular localization of hepatitis C virus envelope glycoprotein complexes expressed by recombinant vaccinia and Sindbis viruses J Viral. 68:6147-60.
19. Duvet, S., L. Cocquerel, A. Pillez, R. Cacan, A. Verbert, D. Moradpour, C. Wychowski, and J. Dubuisson 1998. Hepatitis C virus glycoprotein complex localization in the endoplasmic reticulum involves a determinant for retention and not retrieval J Biol Chem. 273:32088-95.
20. Farci, P., A. Shimoda, D. Wong, T. Cabezon, D. De Gioannis, A. Strazzera, Y. Shimizu, M. Shapiro, H. J. Alter, and R. H. Purcell 1996. Prevention of hepatitis C virus infection in chimpanzees by hyperimmune serum against the hypervariable region 1 of the envelope 2 protein Proc Natl Acad Sci USA. 93: 15394-9.
21. Flint, M., J. Dubuisson, C. Maidens, R. Harrop, G. R. Guile, P. Borrow, and J. A. McKeating 2000. Functional characterization of intracellular and secreted forms of a truncated hepatitis C virus E2 glycoprotein J Viral. 74:702-9.
22. Fournillier, A., E. Depla, P. Karayiannis, O. Vidalin, G. Maertens, C. Trepo, and G. Inchauspe 1999. Expression of noncovalent hepatitis C virus envelope E1-E2 complexes is not required for the induction of antibodies with neutralizing properties following DNA immunization J Virol. 73:7497-504.
23. Geissler, M., K. Tokushige, T. Wakita, V. R. Zurawski, and J. R. Wands 1998. Differential cellular and humoral immune responses to HCV core and HBV envelope proteins after genetic immunizations using chimeric constructs Vaccine. 16:857-67.
24. Gish, R. G. 1999. Standards of treatment in chronic hepatitis C Semin Liver Dis. 19:35-47.
25. Gordon, E. J., R. Bhat, Q. Liu, Y. F. Wang, C. Tackney, and A. M. Prince 2000. Immune responses to hepatitis C virus structural and nonstructural proteins induced by plasmid DNA immunizations J Infect Dis. 181:42-50.
26. Grakoui, A., C. Wychowski, C. Lin, S. M. Feinstone, and C. M. Rice 1993. Expression and identification of hepatitis C virus polyprotein cleavage products J Virol. 67: 1385-95.
27. Hanke, T., T. J. Blanchard, J. Schneider, G. S. 099, R. Tan, M. Becker, S. C. Gilbert, A. V. Hill, G. L. Smith, and A. McMichael 1998. Immunogenicities of intravenous and intramuscular administrations of modified vaccinia virus Ankara-based multi-CTL epitope vaccine for human immunodeficiency virus type 1 in mice J Gen Virol. 79:83-90.
28. Harada, S., Y. Watanabe, K. Takeuchi, T. Suzuki, T. Katayama, Y. Takebe, I. Saito, and T. Miyamura 1991. Expression of processed core protein of hepatitis C virus in mammalian cells J Virol. 65:3015-21.
29. Hirsch, V. M., T. R. Fuerst, G. Sutter, M. W. Carroll, L. C. Yang, S. Goldstein, M. Piatak, Jr., W. R. Elkins, W. G. Alvord, D. C. Montefiori, B. Moss, and J. D. Lifson 1996. Patterns of viral replication correlate with outcome in simian immunodeficiency virus (SIV)-infected macaques: effect of prior immunization with a trivalent SIV vaccine in modified vaccinia virus Ankara J Virol. 70:3741-52.
30. Hollinger 1990. Non-A, non-B hepatitis viruses. Raven Press Ltd, New York.
31. Hoofnagle, J. H., and A. M. di Bisceglie 1997. The treatment of chronic viral hepatitis N Engl J Med. 336:347-56.
32. Inchauspe, G., L. Vitvitski, M. E. Major, G. Jung, U. Spengler, M. Maisonnas, and C. Trepo 1997. Plasmid DNA expressing a secreted or a nonsecreted form of hepatitis C virus nucleocapsid: comparative studies of antibody and T-helper responses following genetic immunization DNA Cell Biol. 16:185-95.
33. Iwarson, S., E. Tabor, H. C. Thomas, P. Snoy, and R. J. Gerety 1985. Protection against hepatitis B virus infection by immunization with hepatitis B core antigen Gastroenterology. 88:763-7.
34. Kato, N., Y. Ootsuyama, H. Sekiya, S. Ohkoshi, T. Nakazawa, M. Hijikata, and K. Shimotohno 1994. Genetic drift in hypervariable region 1 of the viral genome in persistent hepatitis C virus infection J Virol. 68:4776-84.
35. Kuo, G., Q. L. Choo, H. J. Alter, G. L. Gitnick, A. G. Redeker, R. H. Purcell, T. Miyamura, J. L. Dienstag, M. J. Alter, C. E. Stevens, and et al. 1989. An assay for circulating antibodies to a major etiologic virus of human non-A, non-B hepatitis Science. 244:362-4.
36. Large, M. K., D. J. Kittlesen, and Y. s. Hahn 1999. Suppression of host immune response by the core protein of hepatitis C virus: possible implications for hepatitis C virus persistence J Immunol. 162:931-8.
37. Lasarte, J. J., F. J. Corrales, N. Casares, A. Lopez-Diaz de Ceria, C. Qian, X. Xie, F. Borras-Cuesta, and J. Prieto 1999. Different doses of adenoviral vector expressing IL-12 enhance or depress the immune response to a coadministered antigen: the role of nitric oxide J Immunol. 162:5270-7.
38. Li, L, J. He, and L. Zhao 1998. [Epidemiologic features of viral hepatitis in Fujian] Zhonghua Liu Xing Bing Xue Za Zhi. 19:89-92.
39. Liang, T. J. 1998. Combination therapy for hepatitis C infection N Engl J Med. 339:1549-50.
40. Liang, T. J., B. Rehermann, L. B. Seeff, and J. H. Hoofnagle 2000. Pathogenesis, natural history, treatment, and prevention of hepatitis C Ann Intern Med. 132:296-305.
41. Makimura, M., S. Miyake, N. Akino, K. Takamori, Y. Matsuura, T. Miyamura, and I. Saito 1996. Induction of antibodies against structural proteins of hepatitis C virus in mice using recombinant adenovirus Vaccine. 14:28-36.
42. Mayr, A., H. Stickl, H. K. Muller, K. Danner, and H. Singer 1978. [The smallpox vaccination strain MVA: marker, genetic structure, experience gained with the parenteral vaccination and behavior in organisms with a debilitated defence mechanism (author's transl)] Zentralbl Bakteriol [8]. 167:375-90.
43. McLauchlan, J. 2000. Properties of the hepatitis C virus core protein: a structural protein that modulates cellular processes J Viral Hepat. 7:2-14.
44. Men, R., L. Wyatt, I. Tokimatsu, S. Arakaki, G. Shameem, R. Elkins, R. Chanock, B. Moss, and C. J. Lai 2000. Immunization of rhesus monkeys with a recombinant of modified vaccinia virus Ankara expressing a truncated envelope glycoprotein of dengue type 2 virus induced resistance to dengue type 2 virus challenge Vaccine. 18:3113-22.
45. Meyer, H., G. Suffer, and A. Mayr 1991. Mapping of deletions in the genome of the highly attenuated vaccinia virus MVA and their influence on virulence J Gen Virol. 72:1031-8.
46. Michalak, J. P., C. Wychowski, A. Choukhi, J. C. Meunier, S. Ung, C. M. Rice, and J. Dubuisson 1997. Characterization of truncated forms of hepatitis C virus glycoproteins J Gen Virol. 78:2299-306.
47. Mosse, C. A., L. Meadows, C. J. Luckey, D. J. Kittlesen, E. L. Huczko, C. L. Slingluff, J. Shabanowitz, D. F. Hunt, and V. H. Engelhard 1998. The class I antigen-processing pathway for the membrane protein tyrosinase involves translation in the endoplasmic reticulum and processing in the cytosol J Exp Med. 187:37-48.
48. Patel, J., A. H. Patel, and J. McLauchlan 2001. The transmembrane domain of the hepatitis C virus E2 glycoprotein is required for correct folding of the E1 glycoprotein and native complex formation Virology. 279:58-68.
49. Ralston, R., K. Thudium, K. Berger, C. Kuo, B. Gervase, J. Hall, M. Selby, G. Kuo, M. Houghton, and Q. L. Choo 1993. Characterization of hepatitis C virus envelope glycoprotein complexes expressed by recombinant vaccinia viruses J Virol. 67:6753-61.
50. Ramirez, J. C., M. M. Gherardi, and M. Esteban 2000. Biology of attenuated modified vaccinia virus Ankara recombinant vector in mice: virus fate and activation of B- and T-cell immune responses' in comparison with the Western Reserve strain and advantages as a vaccine J Virol. 74:923-33.
51. Santolini, E., G. Migliaccio, and N. La Monica 1994. Biosynthesis and biochemical properties of the hepatitis C virus core protein J Virol. 68:3631-41.
52. Schneider, J., S. C. Gilbert, T. J. Blanchard, T. Hanke, K. J. Robson, C. M. Hannan, M. Becker, R. Sinden, G. L. Smith, and A. V. Hill 1998. Enhanced immunogenicity for CD8+T cell induction and complete protective efficacy of malaria DNA vaccination by boosting with modified vaccinia virus Ankara Nat Med. 4:397-402,
53. Seef, L. B. 1999. Natural history of hepatitis C The American Journal of Medicine. 107:10-15.
54. Selby, M., A. Erickson, C. Dong, S. Cooper, P. Parham, M. Houghton, and C. M. Walker 1999. Hepatitis C virus envelope glycoprotein E1 originates in the endoplasmic reticulum and requires cytoplasmic processing for presentation by class I MHC molecules J Immunol. 162:669-76.
55. Selby, M. J., Q. L. Choo, K. Berger, G. Kuo, E. Glazer, M. Eckart, C. Lee, D. Chien, C. Kuo, and M. Houghton 1993. Expression, identification and subcellular localization of the proteins encoded by the hepatitis C viral genome J Gen Virol. 74:1103-13.
56. Shimizu, Y. K., H. Igarashi, T. Kiyohara, T. Cabezon, P. Farci, R. H. Purcell, and H. Yoshikura 1996. A hyperimmune serum against a synthetic peptide corresponding to the hypervariable region 1 of hepatitis C virus can prevent viral infection in cell cultures Virology. 223:409-12.
57. Shirai, M., H. Okada, M. Nishioka, T. Akatsuka, C. Wychowski, R. Houghten, C. D. Pendleton, S. M. Feinstone, and J. A. Berzofsky 1994. An epitope in hepatitis C virus core region recognized by cytotoxic T cells in mice and humans J Virol. 68:3334-42.
58. Staib, C., I. Drexler, M. Ohlmann, S. Wintersperger, V. Erfle, and G. Sutter 2000. Transient host range selection for genetic engineering of modified vaccinia virus Ankara Biotechniques. 28:1137-42, 1144-6, 1148.
59. Stittelaar, K. J., L. S. Wyatt, R. L. de Swart, H. W. Vos, J. Groen, G. van Amerongen, R. S. van Binnendijk, S. Rozenblatt, B. Moss, and A. D. Osterhaus 2000. Protective immunity in macaques vaccinated with a modified vaccinia virus Ankara-based measles virus vaccine in the presence of passively acquired antibodies J Viral. 74:4236-43.
60. Sutter, G., and B. Moss 1992. Nonreplicating vaccinia vector efficiently expresses recombinant genes Proc Natl Acad Sci USA. 89:10847-51.
61. Sutter, G., L. S. Wyatt, P. L. Foley, J. R. Bennink, and B. Moss 1994. A recombinant vector derived from the host range-restricted and highly attenuated MVA strain of vaccinia virus stimulates protective immunity in mice to influenza virus Vaccine. 12:1032-40.
62. Ulmer, J. B., J. J. Donnely, S. E. Parker, G. H. Rhodes, P. L. Feigner, V. J. Dwarki, S. H. Gromkowski, R. R. Deck, C. M. DeWitt, A. Friedman, and et al. 1993. Heterologous protection against influenza by injection of DNA encoding a viral protein Science. 259:1745-9.
63. Wang, Y., H. Okamoto, F. Tsuda, R. Nagayama, Q. M. Tao, and S. Mishiro 1993. Prevalence, genotypes, and an isolate (HC-C2) of hepatitis C virus in Chinese patients with liver disease J Med Virol. 40:254-60.
64. Wyatt, L. S., S. T. Shors, B. R. Murphy, and B. Moss 1996. Development of a replication-deficient recombinant vaccinia virus vaccine effective against parainfluenza virus 3 infection in an animal model Vaccine. 14:1451-8.
65. Wyatt, L. S., S. S. Whitehead, K. A. Venanzi, B. R. Murphy, and B. Moss 1999. Priming and boosting immunity to respiratory syncytial virus by recombinant replication-defective vaccinia virus MVA Vaccine. 18:392-7.
66. Yasui, K., T. Wakita, K. Tsukiyama-Kohara, S. I. Funahashi, M. Ichikawa, T. Kajita, D. Moradpour, J. R. Wands, and M. Kohara 1998. The native form and maturation process of hepatitis C virus core protein J Virol. 72:6048-55.
67. Zibert, A., H. Meisel, W. Kraas, A. Schulz, G. Jung, and M. Roggendorf 1997. Early antibody response against hypervariable region 1 is associated with acute self-limiting infections of hepatitis C virus Hepatology. 25:1245-9.
68. Zibert, A., E. Schreier, and M. Roggendorf 1995. Antibodies in human sera specific to hypervariable region 1 of hepatitis C virus can block viral attachment Virology. 208:653-61.

What is claimed is:

1. A recombinant modified vaccinia virus Ankara (MVA), wherein the recombinant MVA comprises DNA sequences encoding structural antigens selected from among a hepatitis C virus (HCV) capsid protein core polypeptide, an HCV envelope glycoprotein E1 polypeptide, and an HCV envelope glycoprotein E2 polypeptide, epitopes thereof, and combinations thereof.

2. The recombinant MVA of claim 1, wherein the recombinant MVA comprises DNA sequences encoding an HCV envelope glycoprotein E1 polypeptide, an HCV envelope glycoprotein E2 polypeptide, epitopes thereof, and combinations thereof.

3. The recombinant MVA of claim 2, wherein the DNA sequences encoding the HCV envelope glycoprotein E2 polypeptide encode a mutated form of the HCV envelope glycoprotein E2 polypeptide.

4. The recombinant MVA of claim 3, wherein the mutated form of the HCV envelope glycoprotein E2 polypeptide is a secretable form in which at least a part of the lipophilic portions of the HCV envelope glycoprotein E2 polypeptide is deleted.

5. The recombinant MVA of claim 1, wherein the DNA sequences are integrated into non-essential regions in the MVA genome.

6. The recombinant MVA of claim 1, wherein the DNA sequences are integrated into portions of naturally occurring deletions in the MVA genome.

7. The recombinant MVA of claim 6, wherein the naturally occurring deletion is deletion III or another deletion in a non-essential region of the MVA genome.

8. The recombinant MVA of claim 1, wherein the DNA sequences are under transcriptional control of a promoters selected from vaccinia virus specific promoters and promoters which are not derived from vaccinia virus.

9. The recombinant MVA of claim 8, wherein the DNA sequences are under transcriptional control of the vaccinia virus specific Early/Late promoter P7.5.

10. The recombinant MVA of claim 9, wherein the DNA sequence is under transcriptional control of an enhancer.

11. The recombinant MVA of claim 1, wherein the recombinant MVA encodes HCV E1 and E2 polypeptides that are not able to form heterodimers.

12. A pharmaceutical composition comprising at least one recombinant MVA of claim 1 and pharmaceutically acceptable carriers or adjuvants.

13. The pharmaceutical composition of claim 12, in the form of a vaccine.

14. An isolated eukaryotic cell infected with a recombinant MVA of claim 1.

15. The isolated eukaryotic cell of claim 14, wherein the cell is a chicken embryo fibroblast cell, a baby hamster kidney cell, or an antigen presenting cell.

16. The isolated eukaryotic cell of claim 15, wherein the baby hamster kidney cell is a BHK21 cell.

17. The isolated eukaryotic cell of claim 15, wherein the antigen presenting cell is a dendritic cell.

18. A method for the preparation of recombinant MVA according to claim 1, with the following steps of:
(a) introducing DNA sequences as defined in claim 1, or functional portions or epitopes thereof into a non-essential region of a MVA vector for the preparation of a recombinant MVA vector;
(b) introducing the recombinant MVA vector into a eukaryotic cell and amplifying the vector in said cell; and
(c) optionally isolating virus particles or the DNA or RNA thereof.

19. An isolated nucleic acid molecule, wherein the isolated nucleic acid molecule comprises a DNA of the recombinant MVA of claim 1 or an RNA encoded thereby.
